(19) United States
(12) Patent Application Publication
(10) Pub. No.: US 2022/0051757 A1
(43) Pub. Date: Feb. 17, 2022

(54) REAL-TIME SERIES FORMULAS FOR ONE- AND TWO-COMPARTMENT PHARMACOKINETIC MODELS

(71) Applicant: Michalakis Savva, Savannah, GA (US)

(72) Inventor: Michalakis Savva, Savannah, GA (US)

(21) Appl. No.: 16/992,658

(22) Filed: Aug. 13, 2020

(51) Int. Cl. G16C 20/30; G06N 7/00; G16B 5/00

(52) U.S. Cl. CPC G16C 20/30; G16B 5/00; G06N 7/005

(57) ABSTRACT

The present invention discloses series formulas that can calculate various pharmacokinetic parameters upon administration of multiple maintenance doses and a method to custom-build series equations for dosage regimens that include administration of other doses in addition to the maintenance doses via different routes of administration as a function of dose number and total time in the one- and two-compartment pharmacokinetic models. These series formulas, when compared to the old traditional equations that relate drug concentration to the “dummy” time variable of dosing interval, are ideal for studying time-dependent pharmacokinetic parameters such as the peak time (tₘₐₓ) after multiple extravascular dosages and they offer an alternative elegant way of writing algorithms for calculating and plotting pharmacokinetic parameters as a function of total time.

### BACKGROUND OF THE INVENTION

Mathematical treatments of linear mammillary pharmacokinetic models along with some analytical solutions of drug amount or concentration appeared in the literature in the early 1970 (Wagner, 1981; Benet, 1972, Dubois, 2011). Since then, all mathematical expressions relate to either steady state conditions or time within the first dosing interval (Dipiro et al., 2005). During the last 80+ years there has never been an effort to develop series formulas of drug amount or concentration after administration of multiple doses as a function of real or total time. The present invention relates to a set of mathematical series formulas that can be used to calculate drug amount or concentration upon administration of multiple drug doses of same or different size, via different administration routes as a function of dose number and total time in a one- and two-compartment pharmacokinetic models. The advantage of these real-time series equations as compared to their “dummy” time dosing interval variable counterparts are multiple: First, series equations that allow assessment of pharmacokinetic parameters for dosage regimens that include multiple doses of different size can be custom-produced. Second, time-dependent pharmacokinetic parameters such as peak time after administration of multiple extravascular doses can be simulated as a function of dose number and real time. Third, the area under the curve (AUC) and the average drug concentration can be calculated analytically with definite integration of the series formulas. Fourth, computer programs can be written with nested iterative loops using both dose number and real time (example “program_one_comp_nl_mod”). Fifth, graphs of drug concentration as a function of total time can be plotted without additional lines of code that convert dosing interval into total time.

### SUMMARY OF THE INVENTION

The present invention was conceived when the inventor realized that the absorption and elimination rate- and by extension time-dependent variable peak time (tₘₐₓ) after multiple oral doses could not be studied as a function of dose number because all available pharmacokinetic formulas are expressed as a function of dosing interval and not real time. Time-dependent parameters can best be monitored with equations that describe time. In addition, having formulas written in terms of dosing intervals snakes it harder and require additional line of codes to plot drug concentration as a function of time in multiple dosing regimens. The present invention relates to series formulas that can be used to determine drug amount, concentration, area under the curve (AUC) and peak time and peak concentration after administration of multiple drug doses as IV bolus, extravascularly or orally and by intermittent IV infusion (IIV), as a function of real time in a one- and two-compartment pharmacokinetic models. The present invention also describes a method that produces custom-build series equations that can calculate pharmacokinetic parameters as a function of dose number and real time for dosage regimens that include administration of other doses, different in size from the maintenance doses.

### BRIEF DESCRIPTION OF DRAWINGS

FIG. 1 Two-compartment mammillary models

### DETAILED DESCRIPTION OF THE INVENTION

The approach for each route of administration and compartment model is as follows:

1. Obtain analytical solutions to differential equations

2. Derive the drug amount or concentration terms of a sequence after administration of multiple doses by applying the principle of superposition

3. Derive the pattern of the sequence

4. Determine the partial sums of the series

5. Derive final series formula of drug amount or concentration as a function of dose number and total time

6. Integrate the series formula to derive AUC and average concentration formulas

7. Differentiate the series formula with respect e to derive peak time (tₘₐₓ) as a function of dose number and use it to obtain series formulas for peak concentration as a function of real time after administration of multiple oral or extravascular drug doses that are characterized by first-order absorption rate kinetics.

8. If multiple doses of different size than the maintenance dose are administered at different times, modify series formulas by reducing the dose number index n by the number of different size doses administered prior to the maintenance doses and by adding terms outside of the series maintenance dose summation formula that account for the drug concentration due to the administration of the other doses.

### A. One-Compartment Model

### I. Intermittent IV Infusion (IIV)

1. Differential equations for inputs and outputs:
Input: Drug administered by constant IV infusion (zero-order kinetics
Output: First-order kinetics

$$\frac{d x}{d t} = {k}_{0} - k \cdot x ; x ( 0 ) = 00 \leq t \leq T \\ \frac{d x}{d t} = - k \cdot x ; x ( T ) = x ( from input ) T \leq t \leq τ \qquad$$

Analytical solutions:
During Inputs (0≤t≤T):
Using the integrating factor method and the initial condition,

$$x ( t ) = \frac{{k}_{0}}{k} \cdot ( 1 - {e}^{- k \cdot t} )$$

$$C ( t ) = \frac{{k}_{0}}{CL} \cdot ( 1 - {e}^{- k \cdot t} ) \qquad where C = \frac{x}{V} and \qquad CL = k \cdot V$$

When drug infusion has finished (T≤t≤τ):

x(t)=c·e^{-k·(t-T)}; where c is a constant of integration

$$x ( T ) = \frac{{k}_{0}}{k} \cdot ( 1 - {e}^{- k \cdot T} )$$

$$k ( t ) = \frac{{k}_{0}}{k} \cdot ( 1 - {e}^{- k \cdot T} ) \cdot {e}^{- k \cdot ( t - T )}$$

$$C ( t ) = \frac{{k}_{0}}{CL} \cdot ( 1 - {e}^{- k \cdot T} ) \cdot {e}^{- k \cdot ( t - T )} = C ( T ) \cdot {e}^{- k \cdot ( t - T )} = {C}_{max} \cdot {e}^{- k \cdot ( t - T )}$$

2. Sequence terms for multiple doses administered with a dosing interval τ.
Notice that drug concentration during infusion interval is expressed in terms of the minimum concentration from the previous dose whereas drug concentration during the elimination phase is expressed in terms of the maximum drug concentration at the end of the infusion dose. We are also going to call the drug concentration during infusion (0≤t≤T) as Cₐ and during the pure elimination phase (T≤t≤τ) when infusion has stopped as Cₑ. The terms of the sequence during the first two infusions are:
During the first infusion (n=1):

$$\begin{matrix}{C}_{a , 1} = \frac{{k}_{0}}{CL} \cdot ( 1 - {e}^{- k \cdot t} ) & 0 \leq t \leq T \\ {C}_{max , 1} = \frac{{k}_{0}}{CL} \cdot ( 1 - {e}^{- k \cdot T} ) & t = T \\ {C}_{e , 1} = \frac{{k}_{0}}{CL} \cdot ( 1 - {e}^{- k \cdot T} ) \cdot {e}^{- k \cdot ( t - T )} & T \leq t \leq τ \\ {C}_{min , 1} = \frac{{k}_{0}}{CL} \cdot ( 1 - {e}^{- k \cdot T} ) \cdot {e}^{- k \cdot ( τ - T )} & t = τ\end{matrix}$$

During the second infusion (n=2):

$$\begin{matrix}{C}_{a , 2} = {C}_{min , 1} \cdot {e}^{- k \cdot ( t - τ )} + \frac{{k}_{0}}{CL} \cdot ( 1 - {e}^{- k \cdot ( t - τ )} ) & τ \leq t \leq τ + T \\ {C}_{max , 2} = \frac{{k}_{0}}{CL} \cdot ( 1 - {e}^{- k \cdot T} ) ( 1 + {e}^{- k \cdot τ} ) & t = τ + T \\ {C}_{e , 2} = \frac{{k}_{0}}{CL} \cdot ( 1 - {e}^{- k \cdot T} ) ( 1 + {e}^{- k \cdot τ} ) \cdot {e}^{- k \cdot ( t - τ - T )} & τ + T \leq t \leq 2 τ \\ {C}_{min , 2} = \frac{{k}_{0}}{CL} \cdot ( 1 - {e}^{- k \cdot T} ) \cdot ( 1 + {e}^{- k \cdot τ} ) {e}^{- k \cdot ( τ - T )} & t = 2 τ\end{matrix}$$

### 3. Pattern of Sequence

$$\{ {C}_{max , n} \} = \{ \frac{{k}_{0}}{CL} \cdot ( 1 - {e}^{- k \cdot T} ) \cdot {e}^{- k \cdot ( ( n - 1 ) \cdot τ )} \}$$

$$\{ {C}_{min , n} \} = \{ \frac{{k}_{0}}{CL} \cdot ( 1 - {e}^{- k \cdot T} ) \cdot {e}^{- k \cdot ( n \cdot τ - T )} \}$$

$$\{ {C}_{a , n} \} = \{ {C}_{min , ( n - 1 )} \cdot {e}^{- k \cdot ( t - ( n - 1 ) \cdot τ )} + \frac{{k}_{0}}{CL} \cdot ( 1 - {e}^{- k \cdot ( t - ( n - 1 ) \cdot τ )} ) \} ( n - 1 ) \cdot τ \leq t \leq ( n - 1 ) \cdot τ + T$$

$$\{ {C}_{e , n} \} = \{ {C}_{max , n} \cdot {e}^{- k \cdot ( t - ( n - 1 ) \cdot τ - T )} \} \qquad ( n - 1 ) \cdot τ + T \leq t \leq n \cdot τ$$

4-5. Partial Sums and final formula of Series

$${C}_{max , n} = {\sum }_{n = 1}^{∞} \frac{{k}_{0}}{CL} \cdot ( 1 - {e}^{- k \cdot T} ) \cdot {e}^{- k \cdot ( ( n - 1 ) \cdot τ )} = \frac{{k}_{0}}{CL} \cdot ( 1 - {e}^{- k \cdot T} ) \cdot ( 1 + {e}^{- k \cdot τ} + … + {e}^{- k \cdot ( ( n - 1 ) \cdot τ )} )$$

Multiplying and diving the above by (1−e^{-k·τ}),

$${C}_{max , n} = \frac{{k}_{0}}{CL} \cdot ( 1 - {e}^{- k \cdot T} ) \cdot \frac{( 1 - {e}^{- n \cdot k \cdot τ} )}{( 1 - {e}^{- k \cdot τ} )} \qquad eq . 1$$

Using the same approach,

$$\begin{matrix}{C}_{min , n} = \frac{{k}_{0}}{CL} \cdot ( 1 - {e}^{- k \cdot T} ) \cdot \frac{( 1 - {e}^{- n \cdot k \cdot τ} )}{( 1 - {e}^{- k \cdot τ} )} \cdot {e}^{- k \cdot ( τ - T )} & eq . 2 \\ {C}_{a , n , t} = \frac{{k}_{0}}{CL} \cdot ( 1 - {e}^{- k \cdot T} ) \cdot \frac{( 1 - {e}^{- k \cdot ( n - 1 ) τ} )}{( 1 - {e}^{- k \cdot τ} )} \cdot {e}^{- k \cdot ( t - ( n - 2 ) \cdot τ - T )} + \frac{{k}_{0}}{CL} \cdot ( 1 - {e}^{- k \cdot ( t - ( n - 1 ) \cdot τ )} ) & eq . 3 \\ {C}_{e , n , t} = \frac{{k}_{0}}{CL} \cdot ( 1 - {e}^{- k \cdot T} ) \cdot \frac{( 1 - {e}^{- n \cdot k \cdot τ} )}{( 1 - {e}^{- k \cdot τ} )} \cdot {e}^{- k \cdot ( t - ( n - 1 ) \cdot τ - T )} & eq . 4\end{matrix}$$

6. AUC formulas

$$\begin{matrix}{AUC}_{max , n} = {\int }_{( n - 1 ) \cdot τ}^{( n - 1 ) \cdot τ + T} {C}_{a} dt + {\int }_{( n - 1 ) \cdot τ + T}^{n \cdot τ} {C}_{e} dt & eq . 5 \\ {AUC}_{max , n} = T \cdot \frac{{k}_{0}}{CL} - \frac{{k}_{0}}{k \cdot CL} \cdot ( 1 - {e}^{- k \cdot T} ) \cdot {e}^{- k \cdot ( n \cdot τ - T )} & eq . 6\end{matrix}$$

8. Series Formulas for an initial (n=1) Loading IIV dose (D_{L}) followed by multiple IIV maintenance doses (k₀).

For n=1, use the same series formula (eq. 3-4) but replace k₀ with D_{L}.

$${C}_{a , 1 , t} = \frac{{D}_{L}}{CL} \cdot ( 1 - {e}^{- k \cdot t} )$$

$${C}_{e , 1 , t} = \frac{{D}_{L}}{CL} \cdot ( 1 - {e}^{- k \cdot t} ) \cdot {e}^{- k \cdot ( t - T )}$$

$${C}_{min , 1} = \frac{{D}_{L}}{CL} \cdot ( 1 - {e}^{- k \cdot t} ) \cdot {e}^{- k \cdot ( τ - T )}$$

For n>1, use the same series formula (eq. 3-4) but add C_{min,1} and reduce the index n by the number of loading doses you have administered prior to the maintenance doses (in this case only one D_{L} was administered).

$$\begin{matrix}{C}_{a , n > 1 , t} = \frac{{D}_{L}}{CL} \cdot ( 1 - {e}^{- k \cdot T} ) \cdot {e}^{- k \cdot ( t - T )} + \frac{{k}_{0}}{CL} \cdot ( 1 - {e}^{- k \cdot T} ) \cdot \frac{( 1 - {e}^{- k \cdot ( n - 2 ) \cdot τ} )}{( 1 - {e}^{- k \cdot τ} )} \cdot {e}^{- k \cdot ( t - ( n - 2 ) \cdot τ - T )} + \frac{{k}_{0}}{CL} \cdot ( 1 - {e}^{- k \cdot ( t - ( n - 1 ) \cdot τ )} ) n ∈ [ 2 , ∞ ] & eq . 7 \\ {C}_{e , n , t} = \frac{{D}_{L}}{CL} \cdot ( 1 - {e}^{- k \cdot T} ) \cdot {e}^{- k \cdot ( t - T )} + \frac{{k}_{0}}{CL} \cdot ( 1 - {e}^{- k \cdot T} ) \cdot \frac{( 1 - {e}^{- k \cdot ( n - 1 ) \cdot τ} )}{( 1 - {e}^{- k \cdot τ} )} \cdot {e}^{- k \cdot ( t - ( n - 1 ) \cdot τ - T )} n ∈ [ 1 , ∞ ] & eq . 8\end{matrix}$$

Peak and trough concentrations can be determined from eq. 7 and eq. 8 at t=(n−1)·τ+T and t=n·τ, respectively.

$$\begin{matrix}{C}_{max , n > 1 , t} = \frac{{D}_{L}}{CL} \cdot ( 1 - {e}^{- k \cdot T} ) \cdot {e}^{- k \cdot ( n - 1 ) \cdot τ} + \frac{{k}_{0}}{CL} \cdot ( 1 - {e}^{- k \cdot T} ) \cdot \frac{( 1 - {e}^{- k \cdot ( n - 1 ) \cdot τ} )}{( 1 - {e}^{- k \cdot τ} )} & eq . 9 \\ {C}_{min , n} = \frac{{D}_{L}}{CL} \cdot ( 1 - {e}^{- k \cdot T} ) \cdot {e}^{- k \cdot ( n \cdot \cdot τ - T )} + \frac{{k}_{0}}{CL} \cdot ( 1 - {e}^{- k \cdot T} ) \cdot \frac{( 1 - {e}^{- k \cdot ( n - 1 ) \cdot τ} )}{( 1 - {e}^{- k \cdot τ} )} \cdot {e}^{- k \cdot ( τ - T )} & eq . 10\end{matrix}$$

An example of a Fortran program is shown below:

Module Shared_data
! Purpose:
! To declare data to share between subroutines
implicit none
SAVE
!Declare parameters

| | |
|---|---|
| integer,parameter:: P=120 | ! Infusion time in units of min |
| integer,parameter:: P_h=P/60 | ! Infusion time in units of h |
| integer,parameter:: tau_h=12 | ! Dosing interval in units of h |
| integer,parameter:: tau=720 | ! Dosing interval in units of min |
| real,parameter::DM=596.24 | ! Maintenance dose for dosage interval in units of mg |
| real,parameter:: k0= DM/real(P_h) | ! Maintenance dose infused in mg/h; DM/P_h |
| real,parameter:: k0_min=k0/60.0 | ! Maintenance dose infused in mg/min |
| real,parameter::DL_P=945.00 | ! Loading dose for one dosage interval in units of mg |
| real,parameter:: DL=DL_P/real(P_h) | ! Loading dose infused in units of mg/h; DM/P_h |
| real,parameter:: DL_min=DL/60.00 | ! Loading dose in units of mg/min |
| real,parameter:: CL=2.942 | ! Clearance in L/h |
| real,parameter:: CL_min=0.04903 | ! Clearance in L/min |
| real,parameter:: k_h=0.07783 | ! Elimination rate constant in units h-1 |
| real,parameter:: k=0.001297 | ! Elimination rate constant in min-1 |
| integer,parameter::s_t=20 | ! loop increment in units of min |
| integer,parameter::istart=1 | ! istart of the loop and corresponds to the first infusion |
| integer,parameter::iend=6 | ! iend of the loop and corresponds to the last infusion |
| integer,paratneter :incr=1 | ! increment of the loop in units of n (infusion #) |
| !Declare variables | |
| integer:: n | ! Infusion number (times of injection) |
| integer:: t | ! Time in minutes |
| real:: Ca | ! Drug concentration during infusion (ascending) in mg/L |
| real::Ce | ! Drug concentration after infusion is terminated (descending) in mg/L |
| real::Ca_DL | ! Drug concentration during infusion (ascending phase) in mg/L with loading dose (DL) |
| real::Ce_DL | ! Drug concentration after infusion is terminated with administration of a DL |
| real:: Ca_max=0.0 | ! Maximum drug concentration during infusion (ascending) in mg/L |
| real::Ce_min=0.0 | ! Minimum drug concentration after infusion is terminated (descending) in mg/L |
| real::Ca_max_DL=0.0 | ! Maximum drug concentration during infusion in mg/L with loading dose (DL) |
| real::Ce_min_DL=0.0 | ! Minimum drug concentration after infusion is terminated with administration of a DL |
| real::AUC=0.0 | ! AUC at the end of dosing interval just prior to the infusion of the next dose |
| real::AUC_DL=0.0 | ! AUC at the end of dosing interval just prior to the infusion of the DL in (mg*h)/L |
| real::AUC_ss | ! Steady state or max AUC |

end module Shared_data
program one_comp_nl_mod
! Purpose:
! To calculate non-steady state concentrations as a function of time
! in a one-compartment model with and without a loading dose
! The novel long equations were used - manuscript eq. 37-40 except for the first loop
! For n=1 with DL, I have used the equation derived from superposition principle eq. 52 ascending curve only

| ! Record of revisions: | | |
|---|---|---|
| ! Date | Programmer | Description of change |
| ! [symbol] | [symbol] | [symbol] |
| ! 04/03/20 | M. Savva | Original Code |
| ! 04/06/20 | M. Savva | Loops modified to relate directly to infusion time (P), dosing interval (tau) and constant |

step increment. Nested Loops were adapted to shorten the program and allow increasing
n without writing additional code

USE Shared_data
implicit none

| | |
|---|---|
| | n=1 |
| inf_n_1: | Do t = s_t, P, s:_t |
| | Ca=0.0 |
| | Ca=k0/CL*(1-exp(-k*(rea1(t-(n-1)*tau))))+k0/CL*(1-exp(-k*real(P)))& |
| | *exp(-k*(real(t-P-(n-2)*tau)))*(1-exp(-k*real((n-1)*tau)))/(1-exp(-k*real(tau))) |
| | Ca_DL=0.0 |
| | Ce_DL=0.0 |
| | Ca_DL=Ce_DL*exp(-k*real(t-(n-1)*tau))+DL/CL*(1-exp(-k*real(t-(n-1)*tau))) |
| | write(*,100) ‘n = ’,n,‘t = ’,t,‘C = ’,Ca,‘C_DL = ’,Ca_DL |
| | 100 format (A4,I1,5x,A4,I5,2(5x,A4,F8.4)) |
| | end Do inf_n_1 |
| elim_n_1: | Do t = P+(n-1)*tau, n*tau, s_t |
| | Ce=0.0 |
| | Ce=k0/CL*(1-exp(-k*real(P)))*(1-exp(-k*real(n*tau)))/(1-exp(-k*real(tau)))*exp(-k*real(t-(n-1)*tau-P))) |
| | Ce_DL=0.0 |
| | Ce_DL=DL/CL*(1-exp(-k*real(P)))*exp(-k*(real(t-P)))+k0/CL*(1-exp(-k*real(P)))& |
| | /(1-exp(-k*real(tau)))*exp(-k*(real(t-P-(n-1)*tau)))*(1-exp(-k*real((n-1)*tau))) |
| | write(*,101) ‘n = ’,n,‘t = ’,t,‘C = ’,Ce,‘C_DL = ’,Ce_DL |
| | 101 format (A4,I1,5x,A4,15,2(5x,A4,F8.4)) |
| | end Do elim_n_1 |
| outer: | Do n=2,6 |
| inf_n: | Do t = (n-1)*tau, P+(n-1)*tau, s_t |
| | Ca=0.0 |
| | Ca=k0/CL*(1-exp(-k*(real(t-(n-1)*tau))))+k0/CL*(1-exp(-k*real(P)))& |
| | *exp(-k*(real(t-P-(n-2)*tau)))*(1-exp(-k*real((n-1)*tau)))/(1-exp(-k*real(tau))) |
| | Ca_DL=0.0 |
| | Ca_DL=DL/CL*(1-exp(-k*real(P)))*exp(-k*real(-P+t))+k0/CL*(1-exp(-k*real(P)))/(1-exp(-k*real(tau)))& |
| | *exp(-k*(real(-P+t-(n-2)*tau)))*(1-exp(-k*real((n-2)*tau)))+k0/CL*(1-exp(-k*real(t-(n-1)*tau))) |
| | write(*,102) ‘n = ’,n,‘t = ’,t,‘C = ’,Ca,‘C_DL = ’,Ca_DL |
| | 102 format (A4,I1,5x,A4,I5,2(5x,A4,F8.4)) |
| | end Do inf_n |
| elim_n: | Do t = +P(n-1)*tau, n*tau, s_t |
| | Ce=0.0 |
| | Ce=k0/CL*(1-exp(-k*real(P)))*(1-exp(-k*real(n*tau)))/(1-exp(-k*real(tau)))*exp(-k*(real(t-(n-1)*tau-P))) |
| | Ce_DL=0.0 |
| | Ce_DL=DL/CL*(1-exp(-k*real(P)))*exp(-k*(real(t-P)))+k0/CL*(1-exp(-k*real(P)))& |
| | /(1-exp(-k*real(tau)))*exp(-k*(real(t-P-(n-1)*tau)))*(1-exp(-k*real((n-1)*tau))) |
| | write(*,103) ‘n = ’,n,‘t = ’,t,‘C = ’,Ce,‘C_DL = ’,Ce_DL |
| | 103 format (A4,I1,5x,A4,I5,2(5x,A4,F8.4)) |
| | end Do elim_n |
| | end Do outer |

end program one_comp_nl_d

### II. Multiple IV Bolus Doses

### 1. Differential Equations for Inputs and Outputs:

Input: Drug administered by IV bolus
Output: First-order kinetics

$$\frac{dx}{dt} = - k \cdot x ; \qquad x ( 0 ) = D$$

Analytical solutions:

$$x ( t ) = D \cdot {e}^{- k \cdot t}$$

$$C ( t ) = \frac{D}{V} \cdot {e}^{- k \cdot t}$$

2. Sequence terms for multiple doses administered with a dosing interval τ.

$$\{ {C}_{n} \} = \{ \frac{D}{{V}_{d}} \cdot {e}^{- k \cdot t} , \frac{D}{{V}_{d}} \cdot {e}^{- k \cdot t} + \frac{D}{{V}_{d}} \cdot {e}^{- k \cdot ( t - τ )} , … \}$$

3. Pattern of Sequence

$$\{ {C}_{n} \} = \{ \frac{D}{{V}_{d}} \cdot {e}^{- k \cdot ( t - ( n - 1 ) \cdot τ )} \}$$

Where, t is the total time.

4-5. Partial Sums and final formula of Series

$${C}_{𝔫} = {\sum }_{n = 1}^{∞} \frac{D}{{V}_{d}} \cdot {e}^{- k \cdot ( t - ( n - 1 ) \cdot τ )} = \frac{D}{{V}_{d}} \cdot {e}^{- k \cdot t} \cdot ( 1 + {e}^{k \cdot τ} + … + {e}^{k \cdot ( n - 1 ) \cdot τ} )$$

Multiplying and dividing the above by (1−e^{k·τ}),

$$\begin{matrix}{C}_{n , t} = \frac{D}{{V}_{d}} \cdot \frac{( 1 - {e}^{- k \cdot n \cdot τ} )}{( 1 - {e}^{- k \cdot τ} )} \cdot {e}^{- k \cdot ( t - ( n - 1 ) \cdot τ )} & n , t ∈ [ 0 , ∞ )\end{matrix} \qquad eq . 11$$

Equations for maximum and minimum drug concentration in the absence of loading doses can be derived from eq. 11 by setting t=(n−1)·τ and n·τ, respectively.

$$\begin{matrix}\begin{matrix}{C}_{max , n} = \frac{D}{{V}_{d}} \cdot \frac{( 1 - {e}^{- k \cdot n \cdot τ} )}{( 1 - {e}^{- k \cdot τ} )} & n ∈ [ 0 , ∞ )\end{matrix} & eq . 12 \\ \begin{matrix}{C}_{min , n} = \frac{D}{{V}_{d}} \cdot \frac{( 1 - {e}^{- k \cdot n \cdot τ} )}{( 1 - {e}^{- k \cdot τ} )} \cdot {e}^{- k \cdot τ} & n ∈ [ 0 , ∞ )\end{matrix} & eq . 13\end{matrix}$$

6. AUC formulas

$$\begin{matrix}{AUC}_{n , t} = \frac{D}{{V}_{d}} \cdot \frac{( 1 - {e}^{- k \cdot n \cdot τ} )}{( 1 - {e}^{- k \cdot τ} )} \cdot {\int }_{( n - 1 ) \cdot τ}^{t} {e}^{- k \cdot ( x - ( n - 1 ) \cdot τ )} dx & \\ {AUC}_{n , t} = \frac{D}{CL} \cdot \frac{( 1 - {e}^{- k \cdot n \cdot τ} )}{( 1 - {e}^{- k \cdot τ} )} \cdot ( 1 - {e}^{- k \cdot ( t - ( n - 1 ) \cdot τ )} ) & eq . 14 \\ \begin{matrix}For t = n \cdot τ , {AUC}_{n , t} = {AUC}_{max , n} & ⇒\end{matrix} & \\ {AUC}_{max , n} = \frac{D}{CL} \cdot ( 1 - {e}^{- k \cdot n \cdot τ} ) & eq . 15 \\ {C}_{ave , t} = \frac{1}{( t - ( n - 1 ) \cdot τ )} \cdot \frac{D}{{V}_{d}} \cdot \frac{( 1 - {e}^{- k \cdot n \cdot τ} )}{( 1 - {e}^{- k \cdot τ} )} \cdot {\int }_{( n - 1 ) \cdot τ}^{t} {e}^{- k \cdot ( x - ( n - 1 ) \cdot τ )} dx & eq . 16 \\ {C}_{ave , τ} = \frac{{AUC}_{max , n}}{τ} & eq . 17\end{matrix}$$

8. Series Formulas for an initial (n=1) loading IV bolus (D_{L}) followed by multiple IV maintenance boluses (D).
n=1, use the same series formula eq. 11 but replace D with D_{L}.

$${C}_{1} = \frac{{D}_{L}}{{V}_{d}} \cdot \frac{( 1 - {e}^{- k \cdot n \cdot τ} )}{( 1 - {e}^{- k \cdot τ} )} \cdot {e}^{- k \cdot ( t - ( n - 1 ) \cdot τ )} = \frac{{D}_{L}}{{V}_{d}} \cdot {e}^{- k \cdot t}$$

For n≥2, use the same series formula eq. 11 but add C₁ outside of the summation series formula for the maintenance doses and reduce the index n by the number of loading doses you have administered prior to the maintenance doses (in this case only one D_{L} was administered).

$$\begin{matrix}\begin{matrix}{C}_{n > 1 , t} = \frac{{D}_{L}}{{V}_{d}} \cdot {e}^{- k \cdot t} & + \frac{D}{{V}_{d}} \cdot \frac{( 1 - {e}^{- k \cdot ( n - 1 ) \cdot τ} )}{( 1 - {e}^{- k \cdot τ} )} \cdot {e}^{- k \cdot ( t - ( n - 1 ) \cdot τ )}\end{matrix} & n ∈ [ 2 , ∞ )\end{matrix} t ∈ [ 0 , ∞ ) \qquad eq . 18$$

Equations for maximum and minimum drug concentration in the absence of loading doses can be derived from eq. 18 by setting t=(n−1)·τ and t=n·τ, respectively.

$$\begin{matrix}{C}_{max , n} = \frac{{D}_{L}}{{V}_{d}} \cdot {e}^{- k \cdot ( n - 1 ) \cdot τ} + \frac{D}{{V}_{d}} \cdot \frac{( 1 - {e}^{- k \cdot ( n - 1 ) \cdot τ} )}{( 1 - {e}^{- k \cdot τ} )} & eq . 19 \\ {C}_{min , n} = \frac{{D}_{L}}{{V}_{d}} \cdot {e}^{- k \cdot n \cdot τ} + \frac{D}{{V}_{d}} \cdot \frac{( 1 - {e}^{- k \cdot ( n - 1 ) \cdot τ} )}{( 1 - {e}^{- k \cdot τ} )} \cdot {e}^{- k \cdot τ} & eq . 20\end{matrix}$$

### III. Multiple Extravascular/Oral Doses

1. Differential equations for inputs and outputs:
Input: Drug administered extravascularly with absorption from the site of administration to the central circulation following first-order kinetics
Output: First-order elimination kinetics

$$\frac{d x}{d t} = {k}_{a} \cdot {x}_{v} - k \cdot x ; \qquad x ( 0 ) = 0$$

$$\frac{{dx}_{v}}{d t} = - {k}_{a} \cdot {x}_{v} ; \qquad {x}_{v} ( 0 ) = F \cdot D$$

Analytical solutions:

$$C = \frac{F \cdot D \cdot {k}_{a}}{{V}_{d} \cdot ( {k}_{a} - k )} \cdot ( {e}^{- k \cdot t} - {e}^{- {k}_{a} \cdot t} )$$

$${C}_{v} = \frac{F \cdot D}{{V}_{v}} \cdot {e}^{- {k}_{a} \cdot t}$$

2. Sequence terms for multiple doses administered with a dosing interval τ.

$$\{ {C}_{n} \} = \{ \frac{F \cdot D \cdot {k}_{a}}{{V}_{d} \cdot ( {k}_{a} - k )} \cdot ( {e}^{- k \cdot t} - {e}^{- {k}_{a} \cdot t} ) , \frac{F \cdot D \cdot {k}_{a}}{{V}_{d} \cdot ( {k}_{a} - k )} \cdot ( {e}^{- k \cdot t} - {e}^{- {k}_{a} \cdot t} ) + \frac{F \cdot D \cdot {k}_{a}}{{V}_{d} \cdot ( {k}_{a} - k )} \cdot ( {e}^{- k \cdot ( t - τ )} - {e}^{- {k}_{a} \cdot ( t - τ )} ) , … \}$$

### 3. Pattern of Sequence

$$\{ {C}_{n} \} = \{ \frac{F \cdot D \cdot {k}_{a}}{{V}_{d} \cdot ( {k}_{a} - k )} \cdot ( {e}^{- k \cdot ( t - ( n - 1 ) \cdot τ )} - {e}^{- {k}_{a} \cdot ( t - ( n - 1 ) \cdot τ )} ) \}$$

Where, t is the total time.
4-5. Partial Sums and final formula of Series

$$\begin{matrix}{C}_{n} = {\sum }_{n = 1}^{∞} \frac{F \cdot D \cdot {k}_{a}}{{V}_{d} \cdot ( {k}_{a} - k )} \cdot ( {e}^{- k \cdot ( t - ( n - 1 ) \cdot τ )} - {e}^{- {k}_{a} \cdot ( t - ( n - 1 ) \cdot τ )} ) & \\ {C}_{n , t} = \frac{F \cdot D \cdot {k}_{a}}{{V}_{d} \cdot ( {k}_{a} - k )} \cdot \{ {e}^{- {k}_{a} \cdot ( t + τ )} \cdot \frac{( 1 - {e}^{{k}_{a} \cdot n \cdot τ} )}{( 1 - {e}^{- {k}_{a} \cdot τ} )} - {e}^{- k \cdot ( t + τ )} \cdot \frac{( 1 - {e}^{k \cdot n \cdot τ} )}{( 1 - {e}^{- k \cdot τ} )} \} n , t ∈ [ 0 , ∞ ) \\ or & eq . 21 \\ {C}_{n , t} = \frac{F \cdot D \cdot {k}_{a}}{{V}_{d} \cdot ( {k}_{a} - k )} \cdot \{ {e}^{- k \cdot ( t - ( n - 1 ) \cdot τ )} \cdot \frac{( 1 - {e}^{- k \cdot n \cdot τ} )}{( 1 - {e}^{- k \cdot τ} )} - {e}^{- {k}_{a} \cdot ( t - ( n - 1 ) \cdot τ )} \cdot \frac{( 1 - {e}^{- {k}_{a} \cdot n \cdot τ} )}{( 1 - {e}^{- {k}_{a} \cdot τ} )} \} & eq . 22\end{matrix}$$

The formula for trough drug concentration in the absence of loading doses can be derived from eq. 21 (or eq. 22) at t=n·T.

$${C}_{min , n} = \frac{F \cdot D \cdot {k}_{a}}{{V}_{d} \cdot ( {k}_{a} - k )} \cdot \{ {e}^{- k \cdot τ} \cdot \frac{( 1 - {e}^{- k \cdot n \cdot τ} )}{( 1 - {e}^{- k \cdot τ} )} - {e}^{- {k}_{a} \cdot τ} \cdot \frac{( 1 - {e}^{- {k}_{a} \cdot n \cdot τ} )}{( 1 - {e}^{- {k}_{a} \cdot τ} )} \} \qquad eq . 23$$

6. AUC formulas

$${AUC}_{max , n} = \frac{F \cdot D \cdot {k}_{a}}{{V}_{d} \cdot ( {k}_{a} - k )} \cdot [ \frac{( 1 - {e}^{- k \cdot n \cdot τ} )}{k} - \frac{( 1 - {e}^{- {k}_{a} \cdot n \cdot τ} )}{{k}_{a}} ] \qquad eq . 24$$

7. Differentiate the series formula with respect to time to derive peak real time (tₘₐₓ)and use it to obtain series formulas for peak concentration as a function of real time.

$$\begin{matrix}t = {t}_{max , n} = ( n - 1 ) \cdot τ + \frac{ln ( \frac{{k}_{a}}{k} )}{{k}_{a} - k} + \frac{1}{{k}_{a} - k} \cdot ln ( \frac{( 1 - {e}^{- {k}_{a} \cdot n \cdot τ} )}{( 1 - {e}^{- {k}_{a} \cdot τ} )} \cdot \frac{( 1 - {e}^{- k \cdot τ} )}{( 1 - {e}^{- k \cdot n \cdot τ} )} ) & eq . 25 \\ {C}_{max , n} = \frac{F \cdot D \cdot {k}_{a}}{{V}_{d} \cdot ( {k}_{a} - k )} \cdot \{ {e}^{- {k}_{a} \cdot ( {t}_{max , n} + τ )} \cdot \frac{( 1 - {e}^{{k}_{a} \cdot n \cdot τ} )}{( 1 - {e}^{- {k}_{a} \cdot τ} )} - {e}^{- k \cdot ( {t}_{max , n} + τ )} \cdot \frac{( 1 - {e}^{k \cdot n \cdot τ} )}{( 1 - {e}^{- k \cdot τ} )} \} & eq . 26\end{matrix}$$

8. Series Formulas for an initial (n=1) loading dose (D_{L}) followed by multiple maintenance doses (D) extravascularly.
For n=1, use the same series formula eq. 21 but replace 1) with D_{L}.

$${C}_{1} = \frac{F \cdot {D}_{L} \cdot {k}_{a}}{{V}_{d} \cdot ( {k}_{a} - k )} \cdot ( {e}^{- k \cdot t} - {e}^{- {k}_{a} \cdot t} )$$

For n≥2, use the same series formula eq. 21 but add C₁ outside of the summation series and reduce the index n by the number of loading doses you have administered prior to the maintenance doses (in this case only one D_{L} was administered).

$${C}_{n > 1 , t} = \frac{F \cdot {D}_{L} \cdot {k}_{a}}{{V}_{d} \cdot ( {k}_{a} - k )} \cdot ( {e}^{- k \cdot t} - {e}^{- {k}_{a} \cdot t} ) + \frac{F \cdot D \cdot {k}_{a}}{{V}_{d} \cdot ( {k}_{a} - k )} \cdot \{ {e}^{- k \cdot ( t - ( n - 1 ) \cdot τ )} \cdot \frac{( 1 - {e}^{- k \cdot ( n - 1 ) \cdot τ} )}{( 1 - {e}^{- k \cdot τ} )} - {e}^{- {k}_{a} \cdot ( t - ( n - 1 ) \cdot τ )} \cdot \frac{( 1 - {e}^{- {k}_{a} \cdot ( n - 1 ) \cdot τ} )}{( 1 - {e}^{- {k}_{a} \cdot τ} )} \} n ∈ [ 2 , ∞ ] t ∈ [ 0 , ∞ ) \qquad eq . 27$$

The formulas for peak and trough drug concentration are:

$${C}_{min , n} = \frac{F \cdot {D}_{L} \cdot {k}_{a}}{{V}_{d} \cdot ( {k}_{a} - k )} \cdot ( {e}^{- k \cdot n \cdot τ} - {e}^{- {k}_{a} \cdot n \cdot τ} ) + \frac{F \cdot D \cdot {k}_{a}}{{V}_{d} \cdot ( {k}_{a} - k )} \cdot ( {e}^{- k \cdot τ} \cdot \frac{( 1 - {e}^{- k \cdot ( n - 1 ) \cdot τ} )}{( 1 - {e}^{- k \cdot τ} )} - {e}^{- {k}_{a} \cdot τ} \cdot \frac{( 1 - {e}^{- {k}_{a} \cdot ( n - 1 ) \cdot τ} )}{( 1 - {e}^{- {k}_{a} \cdot τ} )} ) \qquad eq . 28$$

### B. Two-Compartment Mammillary Pharmacokinetic Model

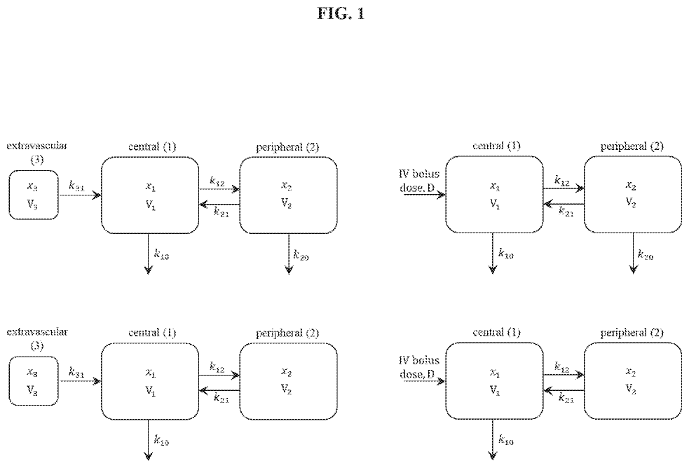

B.1. Multiple Extravascular/Oral Doses with First-Order Absorption Kinetics and First-Order Elimination from Both Central and Peripheral Compartments (FIG. 1, top left panel)
1. Differential equations for inputs and outputs
Input: Drug administered extravascularly/orally with absorption from the site of administration to the central compartment following first-order kinetics
Output: First-order elimination kinetics from central and peripheral compartments

$$\frac{d {x}_{1}}{d t} = - ( {k}_{10} + {k}_{12} ) \cdot {x}_{1} + {k}_{21} \cdot {x}_{2} + {k}_{31} \cdot {x}_{3} ; \qquad {x}_{1} ( 0 ) = 0$$

$$\begin{matrix}\frac{{dx}_{2}}{dt} = {k}_{12} \cdot {x}_{1} - ( {k}_{20} + {k}_{21} ) \cdot {x}_{2} & {x}_{2} ( 0 )\end{matrix} = 0$$

$$\frac{d {x}_{3}}{d t} = - {k}_{31} \cdot {x}_{3} ; \qquad {x}_{3} ( 0 ) = F \cdot D$$

Analytical solutions:

$${C}_{1} ( t ) = \frac{F \cdot D \cdot {k}_{31}}{{V}_{1}} \cdot ( {A}_{1} \cdot {e}^{- {λ}_{1} \cdot t} + {B}_{1} \cdot {e}^{- {λ}_{2} \cdot t} + {E}_{1} \cdot {e}^{- {k}_{31} \cdot t} )$$

$${A}_{1} = \frac{{k}_{20} + {k}_{21} - {λ}_{1}}{( {k}_{31} - {λ}_{1} ) \cdot ( {λ}_{2} - {λ}_{1} )} , \begin{matrix}{B}_{1} = \frac{{k}_{20} + {k}_{21} - {λ}_{2}}{( {k}_{31} - {λ}_{2} ) \cdot ( {λ}_{1} - {λ}_{2} )} , & {E}_{1} = \frac{{k}_{20} + {k}_{21} - {λ}_{31}}{( {k}_{31} - {λ}_{1} ) \cdot ( {λ}_{31} - {λ}_{2} )}\end{matrix}$$

$${λ}_{1} + {λ}_{2} = {k}_{10} + {k}_{12} + {k}_{20} + {k}_{21}$$

$${λ}_{1} \cdot {λ}_{2} = {k}_{10} \cdot {k}_{21} + {k}_{10} \cdot {k}_{20} + {k}_{12} \cdot {k}_{20}$$

$${C}_{2} ( t ) = \frac{F \cdot D \cdot {k}_{31} \cdot {k}_{12}}{{V}_{2}} \cdot ( {A}_{2} \cdot {e}^{- {λ}_{1} \cdot t} + {B}_{2} \cdot {e}^{- {λ}_{2} \cdot t} + {E}_{2} \cdot {e}^{- {k}_{31} \cdot t} )$$

$${A}_{2} = \frac{1}{( {k}_{31} - {λ}_{1} ) \cdot ( {λ}_{2} - {λ}_{1} )} , \begin{matrix}{B}_{2} = \frac{1}{( {k}_{31} - {λ}_{2} ) \cdot ( {λ}_{1} - {λ}_{2} )} , & {E}_{2} = \frac{1}{( {k}_{31} - {λ}_{1} ) \cdot ( {λ}_{31} - {λ}_{2} )}\end{matrix}$$

$${C}_{3} ( t ) = \frac{F \cdot D}{{V}_{3}} \cdot {e}^{- {k}_{31} \cdot t}$$

2. Sequence terms for multiple doses administered with a dosing interval τ.

$$\{ {C}_{1 , n} \} = \{ \frac{F \cdot D \cdot {k}_{31}}{{V}_{1}} \cdot ( {A}_{1} \cdot {e}^{- {λ}_{1} \cdot t} + {B}_{1} \cdot {e}^{- {λ}_{2} \cdot t} + {E}_{1} \cdot {e}^{- {k}_{31} \cdot t} ) , \frac{F \cdot D \cdot {k}_{31}}{{V}_{1}} \cdot ( {A}_{1} \cdot {e}^{- {λ}_{1} \cdot t} + {B}_{1} \cdot {e}^{- {λ}_{2} \cdot t} + {E}_{1} \cdot {e}^{- {k}_{31} \cdot t} ) + \frac{F \cdot D \cdot {k}_{31}}{{V}_{1}} \cdot ( {A}_{1} \cdot {e}^{- {λ}_{1} \cdot ( t - τ )} + {B}_{1} \cdot {e}^{- {λ}_{2} \cdot ( t - τ )} + {E}_{1} \cdot {e}^{- {k}_{31} \cdot ( t - τ )} ) , … \}$$

### 3. Pattern of Sequence

$$\{ {C}_{1 , n} \} = \{ \frac{F \cdot D \cdot {k}_{31}}{{V}_{1}} \cdot ( {A}_{1} \cdot {e}^{- {λ}_{1} \cdot ( t - ( n - 1 ) \cdot τ )} + {B}_{1} \cdot {e}^{- {λ}_{2} \cdot ( t - ( n - 1 ) \cdot τ )} + {E}_{1} \cdot {e}^{- {k}_{31} \cdot ( t - ( n - 1 ) \cdot τ )} ) \}$$

Where, t is the total time.
4-5. Partial Sums and final formula of Series

$$\begin{matrix}{C}_{1 , n} = {\sum }_{n = 1}^{∞} \frac{F \cdot D \cdot {k}_{31}}{{V}_{1}} \cdot ( {A}_{1} \cdot {e}^{- {λ}_{1} \cdot ( t - ( n - 1 ) \cdot τ )} + {B}_{1} \cdot {e}^{- {λ}_{2} \cdot ( t - ( n - 1 ) \cdot τ )} + {E}_{1} \cdot {e}^{- {k}_{31} \cdot ( t - ( n - 1 ) \cdot τ )} ) & \\ {C}_{1 , n , t} = \frac{F \cdot D \cdot {k}_{31}}{{V}_{1}} \cdot \{ {A}_{1} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{1} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} + {E}_{1} \cdot \frac{( 1 - {e}^{- {λ}_{1} \cdot n \cdot τ} )}{( 1 - {e}^{- {λ}_{1} \cdot τ} )} \cdot {e}^{- {k}_{31} \cdot ( t - ( n - 1 ) \cdot τ )} \} n , t ∈ [ 0 , ∞ ) & eq . 30 \\ {C}_{2 , n , t} = \frac{F \cdot D \cdot {k}_{31} \cdot {k}_{12}}{{V}_{2}} \cdot \{ {A}_{2} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{2} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} + {E}_{2} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} \} n , t ∈ [ 0 , ∞ ) & eq . 31\end{matrix}$$

Trough concentration can be obtained from the general equations above at t=n·τ.

$$\begin{matrix}{C}_{1 , min , n} = \frac{F \cdot D \cdot {k}_{31}}{{V}_{1}} \cdot \{ {A}_{1} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{1} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} + {E}_{1} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot τ} \} n ∈ [ 0 , ∞ ] & eq . 32 \\ {C}_{2 , min , n} = \frac{F \cdot D \cdot {k}_{31} \cdot {k}_{12}}{{V}_{1}} \cdot \{ {A}_{2} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{2} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} + {E}_{2} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot τ} \} & eq . 33\end{matrix}$$

6. AUC formulas

$${AUC}_{max , n} = {\int }_{( n - 1 ) \cdot τ}^{n \cdot τ} \frac{F \cdot D \cdot {k}_{31}}{{V}_{1}} \cdot \{ {A}_{2} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{1} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} + {E}_{1} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} \} dt \\ {AUC}_{max , n} = \frac{F \cdot D \cdot {k}_{31}}{{V}_{1}} \cdot [ {A}_{1} \cdot \frac{( 1 - {e}^{- {λ}_{1} \cdot n \cdot τ} )}{{λ}_{1}} + {B}_{1} \cdot \frac{( 1 - {e}^{- {λ}_{2} \cdot n \cdot τ} )}{{λ}_{2}} + {E}_{1} \cdot \frac{( 1 - {e}^{- {k}_{31} \cdot n \cdot τ} )}{{k}_{31}} ] \qquad eq . 34$$

7. Differentiate the series formula with respect to time to derive peak real time (tₘₐₓ) and use it to obtain series formulas for peak concentration as a function of real time.
Due to lack of explicit solutions, the lower eigenvalue exponential term is removed from C_{1,n,t} expression.
Given that E₁=−(A₁+B₁) and assuming that λ₁>λ₂ (if λ₁<λ₂ remove λ₁ exponential term from the eq. 30),

$$\begin{matrix}{C}_{1 , n , t , apr} = \frac{F \cdot D \cdot {k}_{31}}{{V}_{1}} \cdot \{ {A}_{1} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {E}_{1} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} \} \\ \frac{{dC}_{1 , n , t , apr}}{dt} = \frac{F \cdot D \cdot {k}_{31}}{{V}_{1}} \cdot \{ - {λ}_{1} \cdot {A}_{1} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( {t}_{max , n} - ( n - 1 ) \cdot τ )} + {k}_{31} \cdot {E}_{1} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( {t}_{max , n} - ( n - 1 ) \cdot τ )} \} = 0 & \\ {t}_{max , n} = ( n - 1 ) \cdot τ + \frac{ln ( \frac{{k}_{31} \cdot ( {A}_{1} + {B}_{1} ) \cdot ( 1 - {e}^{- {λ}_{1} \cdot τ} ) \cdot ( 1 - {e}^{- {k}_{31} \cdot n \cdot τ} )}{{λ}_{1} \cdot {A}_{1} \cdot ( 1 - {e}^{- {λ}_{1} \cdot n \cdot τ} ) \cdot ( 1 - {e}^{- {k}_{31} \cdot τ} )}}{{k}_{31} - {λ}_{1}} & eq . 35 \\ {C}_{1 , max , n} = \frac{F \cdot D \cdot {k}_{31}}{{V}_{1}} \cdot \{ {A}_{1} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( {t}_{max , n} - ( n - 1 ) \cdot τ )} + {B}_{1} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( {t}_{max , n} - ( n - 1 ) \cdot τ )} + {E}_{1} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( {t}_{max , n} - ( n - 1 ) \cdot τ )} \} & eq . 36\end{matrix}$$

8. Series Formulas for an initial (n=1) loading dose (D_{L}) followed by multiple maintenance doses (D) extravascularly.

For n=1, use the same series formula eq. 30 but replace D with D_{L}.

$${C}_{1} = \frac{F \cdot {D}_{L} \cdot {k}_{31}}{{V}_{1}} \cdot ( {A}_{1} \cdot {e}^{- {λ}_{1} \cdot t} + {B}_{1} \cdot {e}^{- {λ}_{2} \cdot t} + {E}_{1} \cdot {e}^{- {k}_{31} \cdot t} )$$

For n≥2, use the same series formula eq. 30 (or eq. 31 for the peripheral compartment) but add C₁ outside of the summation formula and reduce the index n by the number of loading doses you have administered prior to the maintenance doses (in this case only one D_{L} was administered).

$$\begin{matrix}{C}_{n > 1 , t} = \frac{F \cdot {D}_{L} \cdot {k}_{31}}{{V}_{1}} \cdot ( {A}_{1} \cdot {e}^{- {λ}_{1} \cdot t} + {B}_{1} \cdot {e}^{- {λ}_{2} \cdot t} + {E}_{1} \cdot {e}^{- {k}_{31} \cdot t} ) + \frac{F \cdot D \cdot {k}_{31}}{{V}_{1}} \cdot \{ {A}_{1} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot ( t - ( n - 1 ) \cdot τ )} + {B}_{1} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot ( t - ( n - 1 ) \cdot τ )} + {E}_{1} \cdot \frac{( 1 - {e}^{- {k}_{31} \cdot ( n - 1 ) \cdot τ} )}{( 1 - {e}^{- {k}_{31} \cdot τ} )} \cdot {e}^{- {k}_{31} \cdot ( t - ( n - 1 ) \cdot τ )} \} n ∈ [ 1 , ∞ ] & eq . 37 \\ {C}_{2 , n > 2} = \frac{F \cdot {D}_{L} \cdot {k}_{31} \cdot {k}_{12}}{{V}_{2}} \cdot ( {A}_{2} \cdot {e}^{- {λ}_{1} \cdot t} + {B}_{2} \cdot {e}^{- {λ}_{2} \cdot t} + {E}_{2} \cdot {e}^{- {k}_{31} \cdot t} ) + \frac{F \cdot D \cdot {k}_{31} \cdot {k}_{12}}{{V}_{2}} \cdot \{ {A}_{2} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot ( t - ( n - 1 ) \cdot τ )} + {B}_{2} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot ( t - ( n - 1 ) \cdot τ )} + {E}_{2} \cdot \frac{( 1 - {e}^{- {k}_{31} \cdot ( n - 1 ) \cdot τ} )}{( 1 - {e}^{- {k}_{31} \cdot τ} )} \cdot {e}^{- {k}_{31} \cdot ( t - ( n - 1 ) \cdot τ )} \} & eq . 38\end{matrix}$$

Trough concentration can be obtained from the general equations above at t=n·τ.

$$\begin{matrix}{C}_{1 , min , n} = \frac{F \cdot {D}_{L} \cdot {k}_{31}}{{V}_{1}} \cdot ( {A}_{1} \cdot {e}^{- {λ}_{1} \cdot n \cdot τ} + {B}_{1} \cdot {e}^{- {λ}_{2} \cdot n \cdot τ} + {E}_{1} \cdot {e}^{- {k}_{31} \cdot n \cdot τ} ) + \frac{F \cdot D \cdot {k}_{31}}{{V}_{1}} \cdot \{ {A}_{1} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot τ} + {B}_{1} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot τ} + {E}_{1} \cdot \frac{( 1 - {e}^{- {k}_{31} \cdot ( n - 1 ) \cdot τ} )}{( 1 - {e}^{- {k}_{31} \cdot τ} )} \cdot {e}^{- {k}_{31} \cdot τ} \} n ∈ [ 0 , ∞ ] & eq . 39 \\ {C}_{2 , min , n} = \frac{F \cdot D \cdot {k}_{31} {k}_{12}}{{V}_{2}} \cdot ( {A}_{2} \cdot {e}^{- {λ}_{1} \cdot n \cdot τ} + {B}_{2} \cdot {e}^{- {λ}_{2} \cdot n \cdot τ} + {E}_{2} \cdot {e}^{- {k}_{31} \cdot n \cdot τ} ) + \frac{F \cdot D \cdot {k}_{31} {k}_{12}}{{V}_{2}} \cdot \{ {A}_{2} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot τ} + {B}_{2} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot τ} + {E}_{2} \cdot \frac{( 1 - {e}^{- {k}_{31} \cdot ( n - 1 ) \cdot τ} )}{( 1 - {e}^{- {k}_{31} \cdot τ} )} \cdot {e}^{- {k}_{31} \cdot τ} \} & eq . 40\end{matrix}$$

B.2. Multiple IV Bolus Doses with First-Order Elimination from both Central and Peripheral Compartments (FIG. 1, top right panel)
1. Differential equations for inputs and outputs
Input: Drug administered by an IV bolus directly into the central compartment
Output: First-order elimination kinetics from central and peripheral compartments

$$\frac{d {x}_{1}}{d t} = - ( {k}_{10} + {k}_{12} ) \cdot {x}_{1} + {k}_{21} \cdot {x}_{2} ; \qquad {x}_{1} ( 0 ) = D$$

$$\frac{d {x}_{2}}{d t} = {k}_{12} \cdot {x}_{1} - ( {k}_{20} + {k}_{21} ) \cdot {x}_{2} ; \qquad {x}_{2} ( 0 ) = 0$$

Analytical solutions:

$${C}_{1} ( t ) = \frac{D}{{V}_{1}} \cdot ( {A}_{3} \cdot {e}^{- {λ}_{1} \cdot t} + {B}_{3} \cdot {e}^{- {λ}_{2} \cdot t} )$$

$${A}_{3} = \frac{{k}_{20} + {k}_{21} - {λ}_{1}}{( {λ}_{2} - {λ}_{1} )} , {B}_{3} = \frac{{k}_{20} + {k}_{21} - {λ}_{2}}{( {λ}_{1} - {λ}_{2} )} , \\ {λ}_{1} + {λ}_{2} = {k}_{10} + {k}_{12} + {k}_{20} + {k}_{21} ;$$

$${λ}_{1} \cdot {λ}_{2} = {k}_{10} \cdot {k}_{21} + {k}_{10} \cdot {k}_{20} + {k}_{12} \cdot {k}_{20} + {k}_{12} \cdot {k}_{21}$$

$${C}_{2} ( t ) = \frac{D \cdot {k}_{12}}{{V}_{2} \cdot ( {λ}_{2} - {λ}_{1} )} \cdot ( {e}^{- {λ}_{1} \cdot t} - {e}^{- {λ}_{2} \cdot t} )$$

2. Sequence terms for multiple doses administered with a dosing interval τ.

$$\{ {C}_{1 , n} \} = \{ \frac{D}{{V}_{1}} \cdot ( {A}_{3} \cdot {e}^{- {λ}_{1} \cdot t} + {B}_{3} \cdot {e}^{- {λ}_{2} \cdot t} ) , \frac{D}{{V}_{1}} \cdot ( {A}_{3} \cdot {e}^{- {λ}_{1} \cdot t} + {B}_{3} \cdot {e}^{- {λ}_{2} \cdot t} ) + \frac{D}{{V}_{1}} \cdot ( {A}_{3} \cdot {e}^{- {λ}_{1} \cdot ( t - τ )} - {B}_{3} \cdot {e}^{- {λ}_{2} \cdot ( t - τ )} ) , … \}$$

### 3. Pattern of Sequence

$$\{ {C}_{1 , n} \} = \{ \frac{D}{{V}_{1}} \cdot ( {A}_{3} \cdot {e}^{- {λ}_{1} \cdot ( t - ( n - 1 ) \cdot τ )} + {B}_{3} \cdot {e}^{- {λ}_{2} \cdot ( t - ( n - 1 ) \cdot τ )} ) \}$$

Where, t is the total time.
4-5. Partial Sums and final formula of Series

$$\begin{matrix}{C}_{1 , n} = {\sum }_{n = 1}^{∞} \frac{D}{{V}_{1}} \cdot ( {A}_{3} \cdot {e}^{- {λ}_{1} \cdot ( t - ( n - 1 ) \cdot τ )} + {B}_{3} \cdot {e}^{- {λ}_{2} \cdot ( t - ( n - 1 ) \cdot τ )} ) & \\ {C}_{1 , n , t} = \frac{D}{{V}_{1}} \cdot \{ {A}_{3} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{3} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} \} & eq . 41 \\ {C}_{2 , n , t} = \frac{D \cdot {k}_{12}}{{V}_{2} \cdot ( {λ}_{2} - {λ}_{1} )} \cdot \{ \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} \} & eq . 42\end{matrix}$$

C_{max,n} and C_{min,n} can be obtained from the above formulas at t=(n−1)·τ and t=n·τ, respectively.

$$\begin{matrix}{C}_{1 , max , n} = \frac{D}{{V}_{1}} \cdot \{ {A}_{3} \cdot \frac{( 1 - {e}^{- {λ}_{1} \cdot n \cdot τ} )}{( 1 - {e}^{- {λ}_{1} \cdot τ} )} + {B}_{3} \cdot \frac{( 1 - {e}^{- {λ}_{2} \cdot n \cdot τ} )}{( 1 - {e}^{- {λ}_{2} \cdot τ} )} \} & eq . 43 \\ {C}_{2 , max , n} = \frac{D \cdot {k}_{12}}{{V}_{2} \cdot ( {λ}_{2} - {λ}_{1} )} \cdot \{ \frac{( 1 - {e}^{- {λ}_{1} \cdot n \cdot τ} )}{( 1 - {e}^{- {λ}_{1} \cdot τ} )} + \frac{( 1 - {e}^{- {λ}_{2} \cdot n \cdot τ} )}{( 1 - {e}^{- {λ}_{2} \cdot τ} )} \} & eq . 44 \\ {C}_{1 , min , n} = \frac{D}{{V}_{1}} \{ {A}_{3} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{3} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} \} & eq . 45 \\ {C}_{2 , min , n} = \frac{D \cdot {k}_{12}}{{V}_{2} \cdot ( {λ}_{2} - {λ}_{1} )} \cdot \{ \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} \} & eq .46\end{matrix}$$

6. AUC formulas

$${AUC}_{max , n} = {\int }_{( n - 1 ) \cdot τ}^{n \cdot τ} \frac{D}{{V}_{1}} \cdot \{ {A}_{3} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{3} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} \} dt \\ {AUC}_{max , n} = \frac{D}{{V}_{1}} \cdot [ {A}_{3} \cdot \frac{( 1 - {e}^{- {λ}_{1} \cdot n \cdot τ} )}{{λ}_{1}} + {B}_{3} \cdot \frac{( 1 - {e}^{- {λ}_{2} \cdot n \cdot τ} )}{{λ}_{2}} ] \qquad eq . 47$$

8. Series Formulas for an initial (n=1) loading dose (D_{L}) followed by multiple maintenance doses (D) extravascularly.
For n=1, use the same series formula eq. 41 but replace D with D_{L}.

$${C}_{1} = \frac{{D}_{L}}{{V}_{1}} \cdot ( {A}_{3} \cdot {e}^{- {λ}_{1} \cdot t} + {B}_{3} \cdot {e}^{- {λ}_{2} \cdot t} ) \qquad eq . 48$$

For n≥2, use the same series formula eq. 41 (or eq. 42 for the peripheral compartment) but add C₁ and reduce the index n by the number of loading doses you have administered prior to the maintenance doses (in this case only one D_{L} was administered)

$$\begin{matrix}{C}_{1 , n > 1 , t} = \frac{{D}_{L}}{{V}_{1}} \cdot ( {A}_{3} \cdot {e}^{- {λ}_{1} \cdot t} + {B}_{3} \cdot {e}^{- {λ}_{2} \cdot t} ) + \frac{D}{{V}_{1}} \cdot \{ {A}_{3} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot ( t - ( n - 1 ) \cdot τ )} + {B}_{3} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot ( t - ( n - 1 ) \cdot τ )} \} n ∈ [ 2 , ∞ ) & eq . 49 \\ {C}_{2 , n > 1 , t} = \frac{{D}_{L} \cdot {k}_{12}}{{V}_{2} \cdot ( {λ}_{2} - {λ}_{1} )} \cdot ( {e}^{- {λ}_{1} \cdot t} + {e}^{- {λ}_{2} \cdot t} ) + \frac{D \cdot {k}_{12}}{{V}_{2} \cdot ( {λ}_{2} \cdot {λ}_{1} )} \cdot \{ \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot ( t - ( n - 1 ) \cdot τ )} + \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot ( t - ( n - 1 ) \cdot τ )} \} n ∈ [ 2 , ∞ ) & eq . 50\end{matrix}$$

C_{max,n} and C_{min,n} can be obtained from the above formulas at t=(n−1)·τ and t=n·τ, respectively.

$$\begin{matrix}{C}_{1 , max , n} = \frac{{D}_{L}}{{V}_{1}} \cdot ( {A}_{3} \cdot {e}^{- {λ}_{1} \cdot ( n - 1 ) \cdot τ} + {B}_{3} \cdot ( 1 - {e}^{- {λ}_{2} \cdot ( n - 1 ) \cdot τ} ) + \frac{D}{{V}_{1}} \cdot \{ {A}_{3} \cdot \frac{( 1 - {e}^{- {λ}_{1} \cdot ( n - 1 ) \cdot τ} )}{( 1 - {e}^{- {λ}_{1} \cdot τ} )} + {B}_{3} \cdot \frac{( 1 - {e}^{- {λ}_{2} \cdot ( n - 1 ) \cdot τ} )}{( 1 - {e}^{- {λ}_{2} \cdot τ} )} \} & eq . 51 \\ {C}_{2 , max , n} = \frac{{D}_{L} \cdot {k}_{12}}{{V}_{2} \cdot ( {λ}_{2} - {λ}_{1} )} \cdot ( {e}^{- {λ}_{1} \cdot ( t - ( n - 1 ) \cdot τ )} + {e}^{- {λ}_{2} \cdot ( t - ( n - 1 ) \cdot τ )} ) + \frac{D \cdot {k}_{12}}{{V}_{2} \cdot ( {λ}_{2} - {λ}_{1} )} \cdot \{ \frac{( 1 - {e}^{- {λ}_{1} \cdot ( n - 1 ) \cdot τ} )}{( 1 - {e}^{- {λ}_{1} \cdot τ} )} + \frac{( 1 - {e}^{- {λ}_{2} \cdot ( n - 1 ) \cdot τ} )}{( 1 - {e}^{- {λ}_{2} \cdot τ} )} \} & eq . 52\end{matrix}$$

B.3. Multiple Extravascular/Oral Doses with first-Order Absorption Kinetics and First-Order Elimination only from Central Compartment (FIG. 1, bottom left panel)

1. Differential equations for inputs and outputs

Input: Drug administered extravascularly/orally with absorption from the site of administration to the central compartment following first-order kinetics
Output: First-order elimination kinetics from central compartment

$$\frac{{dx}_{1}}{dt} = - ( {k}_{10} + {k}_{12} ) \cdot {x}_{1} + {k}_{21} \cdot {x}_{2} + {k}_{31} \cdot {x}_{3} ; {x}_{1} ( 0 ) = 0$$

$$\frac{{dx}_{2}}{dt} = {k}_{12} \cdot {x}_{1} - {k}_{21} \cdot {x}_{2} ; {x}_{2} ( 0 ) = 0$$

$$\frac{{dx}_{3}}{dt} = - {k}_{31} \cdot {x}_{3} ; {x}_{3} ( 0 ) = F \cdot D$$

Analytical solutions:

$${C}_{1} ( t ) = \frac{F \cdot D \cdot {k}_{31}}{{V}_{1}} \cdot ( {A}_{4} \cdot {e}^{- {λ}_{1} \cdot t} + {B}_{4} \cdot {e}^{- {λ}_{2} \cdot t} + {E}_{4} \cdot {e}^{- {k}_{31} \cdot t} )$$

$${A}_{4} = \frac{{k}_{21} - {λ}_{1}}{( {k}_{31} - {λ}_{1} ) \cdot ( {λ}_{2} - {λ}_{1} )} ;$$

$${B}_{4} = \frac{{k}_{21} - {λ}_{2}}{( {k}_{31} - {λ}_{2} ) \cdot ( {λ}_{1} - {λ}_{2} )} , \\ {E}_{4} = \frac{{k}_{21} - {k}_{31}}{( {k}_{31} - {λ}_{2} ) \cdot ( {k}_{31} - {λ}_{2} )}$$

$${λ}_{1} + {λ}_{2} = {k}_{10} + {k}_{12} + {k}_{21} ;$$

$${λ}_{1} \cdot {λ}_{2} = {k}_{10} \cdot {k}_{21}$$

$${C}_{2} ( t ) = \frac{F \cdot D \cdot {k}_{31} \cdot {k}_{12}}{{V}_{1}} \cdot ( {A}_{5} \cdot {e}^{- {λ}_{1} \cdot t} + {B}_{5} \cdot {e}^{- {λ}_{2} \cdot t} + {E}_{5} \cdot {e}^{- {k}_{31} \cdot t} )$$

$${A}_{5} = \frac{1}{( {k}_{31} - {λ}_{1} ) \cdot ( {λ}_{2} - {λ}_{1} )} , \\ {B}_{5} = \frac{1}{( {k}_{31} - {λ}_{2} ) \cdot ( {λ}_{1} - {λ}_{2} )} , \\ {E}_{5} = \frac{1}{( {k}_{31} - {λ}_{1} ) \cdot ( {λ}_{31} - {λ}_{2} )} , \\ {C}_{3} ( t ) = \frac{F \cdot D}{{V}_{3}} \cdot {e}^{- {k}_{31} \cdot t}$$

2. Sequence terms for multiple doses administered with a dosing interval τ.

$$\{ {C}_{1 , n} \} = \{ \frac{F \cdot D \cdot {k}_{31}}{{V}_{1}} \cdot ( {A}_{4} \cdot {e}^{- {λ}_{1} \cdot t} + {B}_{4} \cdot {e}^{- {λ}_{2} \cdot t} + {E}_{4} \cdot {e}^{- {k}_{31} \cdot t} ) , \frac{F \cdot D \cdot {k}_{31}}{{V}_{1}} \cdot ( {A}_{4} \cdot {e}^{- {λ}_{1} \cdot t} + {B}_{4} \cdot {e}^{- {λ}_{2} \cdot t} + {E}_{4} \cdot {e}^{- {k}_{31} \cdot t} ) + \frac{F \cdot D \cdot {k}_{31}}{{V}_{1}} \cdot ( {A}_{4} \cdot {e}^{- {λ}_{1} \cdot ( t - τ )} + {B}_{4} \cdot {e}^{- {λ}_{2} \cdot ( t - τ )} + {E}_{4} \cdot {e}^{- {k}_{31} \cdot ( t - τ )} ) , … \}$$

3. Pattern of Sequence

$$\{ {C}_{1 , n} \} = \{ \frac{F \cdot D \cdot {k}_{31}}{{V}_{1}} \cdot ( {A}_{4} \cdot {e}^{- {λ}_{1} \cdot ( t - ( n - 1 ) \cdot τ )} + {B}_{4} \cdot {e}^{- {λ}_{2} \cdot ( t - ( n - 1 ) \cdot τ )} + {E}_{4} \cdot {e}^{- {k}_{31} \cdot ( t - ( n - 1 ) \cdot τ )} ) \}$$

Where, t is the total time.
4-5. Partial Sums and final formula of Series

$$\begin{matrix}{C}_{n} = {\sum }_{n = 1}^{∞} \frac{F \cdot D \cdot {k}_{31}}{{V}_{1}} \cdot ( {A}_{4} \cdot {e}^{- {λ}_{1} \cdot ( t - ( n - 1 ) \cdot τ )} + {B}_{4} \cdot {e}^{- {λ}_{2} \cdot ( t - ( n - 1 ) \cdot τ )} + {E}_{4} \cdot {e}^{- {k}_{31} \cdot ( t - ( n - 1 ) \cdot τ )} ) {C}_{1 , n , t} = \frac{F \cdot D \cdot {k}_{31}}{{V}_{1}} \cdot \{ {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{4} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} + {E}_{4} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} \} n , t ∈ [ 0 , ∞ ) & eq . 55 \\ {C}_{2 , n , t} = \frac{F \cdot D \cdot {k}_{31} \cdot {k}_{12}}{{V}_{2}} \cdot \{ {A}_{5} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{5} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} + {E}_{5} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} \} n , t ∈ [ 0 , ∞ ) & eq . 56 \\ {C}_{3 , n , t} = \frac{F \cdot D}{{V}_{3}} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} n , t ∈ [ 0 , ∞ ) & eq . 57\end{matrix}$$

Trough concentrations can be obtained from the general equations above at t=n·τ

$$\begin{matrix}{C}_{1 , min , n} = \frac{F \cdot D \cdot {k}_{31}}{{V}_{1}} \cdot \{ {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{4} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} + {E}_{4} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot τ} \} n , t ∈ [ 0 , ∞ ) & eq . 58 \\ {C}_{2 , min , n} = \frac{F \cdot D \cdot {k}_{31} \cdot {k}_{12}}{{V}_{2}} \cdot \{ {A}_{5} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} \cdot {B}_{5} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} + {E}_{5} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot τ} \} & eq . 59\end{matrix}$$

6. AUC formulas

$${AUC}_{max , n} = {\int }_{( n - 1 ) \cdot τ}^{n \cdot τ} \frac{F \cdot D \cdot {k}_{31}}{{V}_{1}} \cdot \{ {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{4} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} + {E}_{4} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( t - ( n - 1 ) \cdot τ )} \} dt \\ {AUC}_{max , n} = \frac{F \cdot D \cdot {k}_{31}}{{V}_{1}} \cdot [ {A}_{4} \cdot \frac{( 1 - {e}^{- {λ}_{1} \cdot n \cdot τ} )}{{λ}_{1}} + {B}_{4} \cdot \frac{( 1 - {e}^{- {λ}_{2} \cdot n \cdot τ} )}{{λ}_{2}} + {E}_{4} \cdot \frac{( 1 - {e}^{- {k}_{31} \cdot n \cdot τ} )}{{k}_{31}} ] \qquad eq . 60$$

7. Differentiate the series formula with respect to time to derive peak real time (tₘₐₓ) and use it to obtain series formulas for peak concentration as a function of real time.
Due to lack of explicit solutions, the lower eigenvalue exponential term will be removed from expression.
Given that E₄=−(A₄+B₄) and assuming that λ₁>λ₂ (if λ₁<λ₂ remove λ₁ exponential term from the equation below),

$$\begin{matrix}{C}_{1 , n , t , apr} = \frac{F \cdot D \cdot {k}_{31}}{{V}_{1}} \cdot \{ {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {E}_{4} \cdot \frac{( 1 - {e}^{- {λ}_{31} \cdot n \cdot τ} )}{( 1 - {e}^{- {λ}_{31} \cdot τ} )} \cdot {e}^{- {λ}_{1} \cdot ( t - ( n - 1 ) \cdot τ )} \} \frac{{dC}_{1 , n , t , apr}}{dt} = \frac{F \cdot D \cdot {k}_{31}}{{V}_{1}} \cdot \{ - {λ}_{1} \cdot {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( {t}_{max , n} - ( n - 1 ) \cdot τ )} + {k}_{31} \cdot {E}_{4} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( {t}_{max , n} - ( n - 1 ) \cdot τ )} \} = 0 \\ {t}_{max , n} = ( n - 1 ) \cdot τ + \frac{ln ( \frac{{k}_{31} \cdot ( {A}_{4} + {B}_{4} ) \cdot ( 1 - {e}^{- {λ}_{1} \cdot τ} ) \cdot ( 1 - {e}^{- {k}_{31} \cdot n \cdot τ} )}{{λ}_{1} \cdot {A}_{4} \cdot ( 1 - {e}^{- {λ}_{1} \cdot n \cdot τ} ) \cdot ( 1 - {e}^{- {k}_{31} \cdot τ} )} )}{{k}_{31} - {λ}_{1}} & eq . 61 \\ {C}_{1 , max , n} = \frac{F \cdot D \cdot {k}_{31}}{{V}_{1}} \cdot \{ {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( {t}_{max , n} - ( n - 1 ) \cdot τ )} + {B}_{4} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( {t}_{max , n} - ( n - 1 ) \cdot τ )} + {E}_{4} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot ( {t}_{max , n} - ( n - 1 ) \cdot τ )} \} & eq . 62\end{matrix}$$

8. Series Formulas for an initial (n=1) loading dose (D_{L}followed by multiple maintenance doses (D) extravascularly.
For n=1, use the same series formula eq. 55 but replace D with D_{L}.

$${C}_{1} = \frac{F \cdot {D}_{L} \cdot {k}_{31}}{{V}_{1}} \cdot ( {A}_{4} \cdot {e}^{- {λ}_{1} \cdot t} + {B}_{4} \cdot {e}^{- {λ}_{2} \cdot t} + {E}_{4} \cdot {e}^{- {k}_{31} \cdot t} )$$

For n≥2, use the same series formula eq. 55 (or eq. 56 for the peripheral compartment) but add C_{L} and reduce the index n by the number of loading doses you have administered prior to the maintenance doses (in this case only one D_{L} was administered).

$$\begin{matrix}{C}_{1 , n > 1 , t} = \frac{F \cdot {D}_{L} \cdot {k}_{31}}{{V}_{1}} \cdot ( {A}_{4} \cdot {e}^{- {λ}_{1} \cdot t} + {B}_{4} \cdot {e}^{- {λ}_{2} \cdot t} + {E}_{4} \cdot {e}^{- {k}_{31} \cdot t} ) + \frac{F \cdot D \cdot {k}_{31}}{{V}_{1}} \cdot \{ {A}_{4} \cdot \frac{( 1 - {e}^{- {λ}_{1} \cdot ( n - 1 ) \cdot τ} )}{( 1 - {e}^{- λ \cdot τ} )} \cdot {e}^{- {λ}_{1} \cdot ( t - ( n - 1 ) \cdot τ )} + {B}_{4} \cdot \frac{( 1 - {e}^{- {k}_{2} \cdot ( n - 1 ) \cdot τ} )}{( 1 - {e}^{- {k}_{2} \cdot τ} )} \cdot {e}^{- {k}_{2} \cdot ( t - ( n - 1 ) \cdot τ )} + {E}_{4} \cdot \frac{( 1 - {e}^{- {k}_{31} \cdot ( n - 1 ) \cdot τ} )}{( 1 - {e}^{- {k}_{31} \cdot τ} )} \cdot {e}^{- {k}_{31} \cdot ( t - ( n - 1 ) \cdot τ )} \} n ∈ [ 2 , ∞ ) & eq . 63 \\ {C}_{2 , n > 1 , t} = \frac{F \cdot {D}_{L} \cdot {k}_{31} \cdot {k}_{12}}{{V}_{2}} \cdot ( {A}_{5} \cdot {e}^{- {λ}_{1} \cdot t} + {B}_{5} \cdot {e}^{- {λ}_{2} \cdot t} + {E}_{5} \cdot {e}^{- {k}_{31} \cdot t} ) + \frac{F \cdot D \cdot {k}_{31} \cdot {k}_{12}}{{V}_{2}} \cdot \{ {A}_{5} \cdot \frac{( 1 - {e}^{- {λ}_{1} \cdot ( n - 1 ) \cdot τ} )}{( 1 - {e}^{- λ \cdot τ} )} \cdot {e}^{- {λ}_{1} \cdot ( t - ( n - 1 ) \cdot τ )} + {B}_{5} \cdot \frac{( 1 - {e}^{- {k}_{2} \cdot ( n - 1 ) \cdot τ} )}{( 1 - {e}^{- {k}_{2} \cdot τ} )} \cdot {e}^{- {k}_{2} \cdot ( t - ( n - 1 ) \cdot τ )} + {E}_{5} \cdot \frac{( 1 - {e}^{- {k}_{31} \cdot ( n - 1 ) \cdot τ} )}{( 1 - {e}^{- {k}_{31} \cdot τ} )} \cdot {e}^{- {k}_{31} \cdot ( t - ( n - 1 ) \cdot τ )} \} n , t ∈ [ 0 , ∞ ) & eq . 64\end{matrix}$$

Trough concentrations can be obtained from the general equations above at t=n·τ.

$$\begin{matrix}{C}_{1 , min , n} = \frac{F \cdot D \cdot {k}_{31} \cdot {k}_{12}}{{V}_{2}} \cdot ( {A}_{4} \cdot {e}^{- {λ}_{1} \cdot n \cdot τ} + {B}_{4} \cdot {e}^{- {λ}_{2} \cdot n \cdot τ} + {E}_{4} \cdot {e}^{- {k}_{31} \cdot n \cdot τ} ) + \frac{F \cdot D \cdot {k}_{31}}{{V}_{1}} \cdot \{ {A}_{4} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{4} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} + {E}_{4} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot τ} \} & eq . 65 \\ {C}_{2 , min , n} = \frac{F \cdot D \cdot {k}_{31} \cdot {k}_{12}}{{V}_{2}} \cdot ( {A}_{5} \cdot {e}^{- {λ}_{1} \cdot n \cdot τ} + {B}_{5} \cdot {e}^{- {λ}_{2} \cdot n \cdot τ} + {E}_{5} \cdot {e}^{- {k}_{31} \cdot n \cdot τ} ) + \frac{F \cdot D \cdot {k}_{31} \cdot {k}_{12}}{{V}_{1}} \cdot \{ {A}_{5} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{5} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot τ} + {E}_{5} \cdot \frac{( 1 - {e}^{- {k}_{31} \cdot n \cdot τ} )}{( 1 - {e}^{- {k}_{31} \cdot τ} )} \cdot {e}^{- {k}_{31} \cdot τ} \} & eq . 66\end{matrix}$$

B.4. Multiple IV Bolus Doses with First-Order Elimination only from Central Compartment (FIG. 1, bottom right panel)
1. Differential equations for inputs and outputs
Input: Drug administered by an IV bolus directly into the central compartment
Output: First-order elimination kinetics from central compartment

$$\frac{{dx}_{1}}{dt} = - ( {k}_{10} + {k}_{12} ) \cdot {x}_{1} + {k}_{21} \cdot {x}_{2} ; {x}_{1} ( 0 ) = D$$

$$\frac{{dx}_{2}}{dt} = {k}_{12} \cdot {x}_{1} - {k}_{21} \cdot {x}_{2} ; {x}_{2} ( 0 ) = 0$$

Analytical solutions:

$${C}_{1} ( t ) = \frac{D}{{V}_{1}} \cdot ( {A}_{6} \cdot {e}^{- {λ}_{1} \cdot t} + {B}_{6} \cdot {e}^{- {λ}_{2} \cdot t} )$$

$${A}_{6} = \frac{{k}_{21} - {λ}_{1}}{( {λ}_{2} - {λ}_{1} )} , \\ {B}_{6} = \frac{{λ}_{2} - {k}_{21}}{( {λ}_{2} - {λ}_{1} )} , \\ {λ}_{1} + {λ}_{2} = {k}_{10} + {k}_{12} + {k}_{21}$$

$${λ}_{1} \cdot {λ}_{2} = {k}_{10} \cdot {k}_{21}$$

$${C}_{2} ( t ) = \frac{D \cdot {k}_{12}}{{V}_{2} \cdot ( {λ}_{2} - {λ}_{1} )} \cdot ( {e}^{- {λ}_{1} \cdot t} - {e}^{- {λ}_{2} \cdot t} )$$

2. Sequence terms for multiple doses administered with a dosing interval τ.

$$\{ {C}_{n} \} = \{ \frac{D}{{V}_{1}} \cdot ( {A}_{6} \cdot {e}^{- {λ}_{1} \cdot t} + {B}_{6} \cdot {e}^{- {λ}_{2} \cdot t} ) , \frac{D}{{V}_{1}} \cdot ( {A}_{6} \cdot {e}^{- {λ}_{1} \cdot t} + {B}_{6} \cdot {e}^{- {λ}_{2} \cdot t} ) + \frac{D}{{V}_{1}} \cdot ( {A}_{6} \cdot {e}^{- {λ}_{1} \cdot ( t - τ )} - {B}_{6} \cdot {e}^{- {λ}_{2} \cdot ( t - τ )} ) , … \}$$

### 3. Pattern of Sequence

$$\{ {C}_{1 , n} \} = \{ \frac{D}{{V}_{1}} \cdot ( {A}_{6} \cdot {e}^{- {λ}_{1} \cdot ( t - ( n - 1 ) \cdot τ )} + {B}_{6} \cdot {e}^{- {λ}_{2} \cdot ( t - ( n - 1 ) \cdot τ )} ) \}$$

Where, t is the total time.
4-5. Partial Sums and final formula of Series

$$\begin{matrix} {C}_{1 , n} = {\sum }_{n = 1}^{∞} \frac{D}{{V}_{1}} \cdot ( {A}_{6} \cdot {e}^{- {λ}_{1} \cdot ( t - ( n - 1 ) \cdot τ )} + {B}_{6} \cdot {e}^{- {λ}_{2} \cdot ( t - ( n - 1 ) \cdot τ )} ) \\ {C}_{1 , n , t} = \frac{D}{{V}_{1}} \cdot \{ {A}_{6} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{6} \cdot \frac{( 1 - {e}^{- {λ}_{2} \cdot n \cdot t} )}{( 1 - {e}^{- {λ}_{2} \cdot τ} )} \cdot {e}^{- {λ}_{2} \cdot ( t - ( n - 1 ) \cdot τ )} \} & eq . 67 \\ {C}_{2 , n , t} = \frac{D \cdot {k}_{12}}{{V}_{2} \cdot ( {λ}_{2} - {λ}_{1} )} \cdot \{ \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} - \frac{( 1 - {e}^{- {λ}_{2} \cdot n \cdot t} )}{( 1 - {e}^{- {λ}_{2} \cdot τ} )} \cdot {e}^{- {λ}_{2} \cdot ( t - ( n - 1 ) \cdot τ )} \} & eq . 42\end{matrix}$$

C_{max,n} and C_{min,n} can be obtained from the formulas above at t=(n−1)·τ and t=n·τ, respectively.

$$\begin{matrix} {C}_{1 , max , n} = \frac{D}{{V}_{1}} \cdot \{ {A}_{6} \cdot \frac{( 1 - {e}^{- {λ}_{1} \cdot n \cdot τ} )}{( 1 - {e}^{- {λ}_{1} \cdot τ} )} + {B}_{6} \cdot \frac{( 1 - {e}^{- {λ}_{2} \cdot n \cdot τ} )}{( 1 - {e}^{- {λ}_{2} \cdot τ} )} \} & eq . 68 \\ {C}_{1 , min , n} = \frac{D}{{V}_{1}} \cdot \{ {A}_{6} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot τ} + {B}_{6} \cdot \frac{( 1 - {e}^{- {λ}_{2} \cdot n \cdot τ} )}{( 1 - {e}^{- {λ}_{2} \cdot τ} )} \} & eq . 69\end{matrix}$$

The equations for the peripheral compartment C_{2,max,n} and C_{2,min,n} are the same as those derived in section B2 (eq. 44 and eq. 46).
6. AUC formulas

$${AUC}_{max , n} = {\int }_{( n - 1 ) \cdot τ}^{n \cdot τ} \frac{D}{{V}_{1}} \cdot \{ {A}_{6} \cdot \frac{( 1 - {e}^{- {λ}_{1} \cdot n \cdot τ} )}{( 1 - {e}^{- {λ}_{1} \cdot τ} )} \cdot {e}^{- {λ}_{1} \cdot ( t - ( n - 1 ) \cdot τ )} + {B}_{6} \cdot \frac{( 1 - {e}^{- {λ}_{2} \cdot n \cdot τ} )}{( 1 - {e}^{- {λ}_{2} \cdot τ} )} \cdot {e}^{- {λ}_{2} \cdot ( t - ( n - 1 ) \cdot τ )} \} dt \\ {AUC}_{max , n} = \frac{D}{{V}_{1}} \cdot [ {A}_{6} \cdot \frac{( 1 - {e}^{- {λ}_{1} \cdot n \cdot τ} )}{{λ}_{1}} + {B}_{6} \cdot \frac{( 1 - {e}^{- {λ}_{2} \cdot n \cdot τ} )}{{λ}_{2}} ] \qquad eq . 70$$

8. Series Formulas for an initial (n=1) loading dose (D_{L}) followed by multiple maintenance doses (D) extravascularly.
For n=1, use the same series formula eq. 67 (or eq. 42 for the peripheral compartment) but replace D with D_{L}.

$${C}_{1} = \frac{{D}_{L}}{{V}_{1}} \cdot ( {A}_{6} \cdot {e}^{- {λ}_{1} \cdot τ} + {B}_{6} \cdot {e}^{- {λ}_{2} \cdot t} )$$

For n≥2, use the same series formula eq. 67 but add C₁ and reduce the index n by the number of loading doses you have administered prior to the maintenance doses (in this case only one D_{L} was administered).

$${C}_{1 , n > 1 , t} = \frac{{D}_{L}}{{V}_{1}} \cdot ( {A}_{6} \cdot {e}^{- {λ}_{1} \cdot t} + {B}_{6} \cdot {e}^{- {λ}_{2} \cdot t} ) + \frac{D}{{V}_{1}} \cdot \{ {A}_{6} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot ( t - ( ( n - 1 ) \cdot τ )} + {B}_{6} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot ( t - ( n - 1 ) \cdot τ )} \} n ∈ [ 1 , ∞ ) \qquad eq . 71$$

C_{max,n} and C_{min,n} can be obtained from the above formulas at t=(n−1)·τ and t=n·τ, respectively.

$$\begin{matrix}{C}_{1 , max , n} = \frac{{D}_{L}}{{V}_{1}} \cdot ( {A}_{6} \cdot {e}^{- {λ}_{1} \cdot ( n - 1 ) \cdot τ} + {B}_{6} \cdot {e}^{- {λ}_{2} \cdot ( n - 1 ) \cdot τ} ) + \frac{D}{{V}_{1}} \cdot \{ {A}_{6} \cdot \frac{( 1 - {e}^{- {λ}_{1} \cdot ( n - 1 ) \cdot τ} )}{( 1 - {e}^{- {λ}_{1} \cdot τ} )} + {B}_{6} \cdot \frac{( 1 - {e}^{- {λ}_{2} \cdot ( n - 1 ) \cdot τ} )}{( 1 - {e}^{- {λ}_{2} \cdot τ} )} \} & eq . 72 \\ {C}_{1 , min , n} = \frac{{D}_{L}}{{V}_{1}} \cdot ( {A}_{6} \cdot {e}^{- {λ}_{1} \cdot n \cdot τ} + {B}_{6} \cdot {e}^{- {λ}_{2} \cdot n \cdot τ} ) + \frac{D}{{V}_{1}} \cdot \{ {A}_{6} \cdot \frac{( 1 - {e}^{- {λ}_{1} \cdot ( n - 1 ) \cdot τ} )}{( 1 - {e}^{- {λ}_{1} \cdot τ} )} \cdot {e}^{- {λ}_{1} \cdot τ} + {B}_{6} \cdot \frac{( 1 - {e}^{- {λ}_{2} \cdot ( n - 1 ) \cdot τ} )}{( 1 - {e}^{- {λ}_{2} \cdot τ} )} \cdot {e}^{- {λ}_{2} \cdot τ} \} & eq . 73\end{matrix}$$

The equations for the peripheral compartment are the same as those derived in section B.2 (eq. 50, eq. 52 and eq. 54).

### DEFINITION OF TERMS

x: drug amount
τ: dosing interval in units of time
T: infusion time
k: first-order elimination rate constant
k₀: zero-order rate of drug infusion which is also the maintenance dose (D_{M}) in IIV
V: volume of distribution
D: drug dose
D_{M}: drug maintenance dose
D_{L}: drug loading or booster dose
C_{a,2}: Drug concentration and during the second drug infusion
C_{e,1}: Drug concentration when the first drug infusion is finished during the pure elimination phase
T≤t≤τ
xᵥ: Drug amount in the extravascular compartment r site of administration
kₐ: First-order absorption rate constant
Cᵥ: concentration of the extravascular site of administration
F: bioavailability factor
k₃₁: First-order absorption rate constant in the two-compartment models

### REFERENCES

Benet, L. Z., General treatment of linear mammillary models with elimination from any compartment as used in pharmacokinetics. Pharm. Sci., 61 (4), 536-541,1981.
J. T., Spruill, W. J, Wade, W. E., Blouin, R. A. and Pruemer, J. M., Concepts in Clinical Pharmacokinetics. 2005, American Society of Health-System Pharmacist, MD, USA.
Wagner G J, History of Pharmacokinetics, Pharmac. Ther. 12, 537/-562,1981
Dubois, A., Mathematical expressions of the pharmacokinetic and pharmacodynamic models implemented in the PFIM software. 2011. http:/www.pfim.biostat.fr/PFIM PKPD library.pdf

I claim:

1. The real time series formulas in a one-compartment and two-compartment pharmacokinetic models developed and numbered in this document that can be used to:
execute an algorithm programmed in high- or low-level language to calculate drug amount, concentration, minimum and maximum drug amounts and concentrations, AUC, average drug concentration as the AUC divided by the dosing interval, peak time and peak concentration;
execute an algorithm programmed in a computer language and plot the relationship between the aforementioned calculated pharmacokinetic parameters and as a function of time and dose number.
produce series formulas with rearranged numerators and denominators but are otherwise the same formulas. For example, eq. 21 is the same as eq. 22.

2. A method to custom-build series formulas for administration of other doses that differ in size from the maintenance doses, to calculate all pharmacokinetic parameters described in claim 1 by:
adding the concentration(s) or amounts due to administration of the other doses using the principle of superposition outside of the summation series formulas and subtracting the number of other doses from the index n of the original maintenance dose series formulas.
